(19) United States
(12) Patent Application Publication
(10) Pub. No.: US 2022/0130521 A1
(43) Pub. Date: Apr. 28, 2022

(54) SYSTEM AND METHODS FOR COMMUNICATING AND TRACKING IDENTIFICATION SCRUBBED, MOBILE VIEWABLE MESSAGES CONTAINING IMAGES

(71) Applicant: CERNER INNOVATION, INC., Kansas City, KS (US)

(72) Inventors: Sowmya P, Tirupati (IN); Kiran Bhojaraja, Bengaluru (IN); KarthicKumar A, Salem (IN); Sabhariesh H, Bangalore (IN); Padmapriya S, Hosur (IN)

(21) Appl. No.: 17/082,224

(22) Filed: Oct. 28, 2020

(51) Int. Cl. G16H 30/20; H04L 12/58; G16H 40/20; G16H 30/40; G06Q 30/00; G06Q 50/26; G06Q 10/10; G16H 15/00; G16H 40/67; G06F 16/955; G06F 21/62; H04L 29/06; A61B 5/00

(52) U.S. Cl. CPC G16H 30/20; A61B 5/7425; H04L 51/10; G16H 40/20; G16H 30/40; G06Q 30/0185; G06Q 50/265; G06Q 10/107; G16H 15/00; G16H 40/67; G06F 16/9558; G06F 21/6254; H04L 63/08; A61B 5/0013; H04L 51/08

(57) ABSTRACT

Methods, systems, and computer-readable media are disclosed herein that provide services for automatically facilitate mobile-friendly messages containing identification-scrubbed clinical images transferred between a secure healthcare system and an external application that is accessed on a mobile device. Through the services and using minimal inputs, a scrubbed outgoing message containing patient-specific images is automatically generated and transmitted to a mobile device for a user to review. The services automatically track replies as incoming messages that can be mapped back to one patient and one patient visit using unique identifiers that ensure patient information is not shared outside the secure healthcare system. Also, the services automatically extract clinical finding from incoming messages and provide notifications containing the clinical findings to the appropriate clinician for the patient that corresponds to the reviewed images.

### SUMMARY

In order to review radiology and/or other clinical images, a clinician generally must access an internal healthcare system using a particular application, service, or tools. These particular application, service, or tools, however, are not “mobile friendly” and may not be accessible outside of a desktop computer.

### SUMMARY

This Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter. The present invention is defined by the claims as supported by the Specification, including the Detailed Description.

In brief and at a high level, this disclosure describes, among other things, methods, systems, and computer-readable media for facilitating bi-directional communications between a centralized healthcare system and mobile devices, wherein the communications provide remote, mobile device clinical image viewing and clinical finding reporting while maintaining patient information security standards.

In one aspect, one or more non-transitory computer-readable media having computer-executable instructions embodied thereon are provided that, when executed, perform a method for a sender service. In aspects, a request to transmit an outgoing electronic communication and a designated clinical encounter is received. The outgoing electronic communication is automatically generated in response to the request, and the outgoing electronic communication includes the designated clinical encounter for review by a user, in some aspect. The outgoing electronic communication that includes the designated clinical encounter is automatically transmitted, in aspects. The outgoing electronic communication has an identifier that is associated with the designated clinical encounter, in various aspects.

In another aspect, one or more non-transitory computer-readable media having computer-executable instructions embodied thereon are provided that, when executed, perform a method for a receiver service. In some aspects, a first indication of a request to transmit an outgoing electronic communication and a designated clinical encounter is received. The designated clinical encounter includes one or more digitized images captured for a clinical encounter of a patient, in one aspect. In response to receiving the first indication, a unique identifier is generated for the designated clinical encounter and the unique identifier is free of Patient Identifying Information (PII). In aspects, the unique identifier is assigned to the designated clinical encounter. An association of the designated clinical encounter and the unique identifier assigned to the designated clinical encounter are stored in memory, in some aspects. A second indication is received indicating transmission of the outgoing electronic communication and the designated clinical encounter, in an aspect. In one aspect, in response to the second indication, incoming electronic communications are monitored for inclusion of the unique identifier assigned to the designated clinical encounter. Based on the monitoring, an incoming electronic communication is received from a first clinical user that includes the unique identifier assigned to the designated clinical encounter in the memory, in various aspects. The incoming electronic communication includes, in some, one or more clinical findings from the first clinical user for the designated clinical encounter. In an aspect, the unique identifier is extracted from the incoming electronic communication and mapped to the designated clinical encounter based on the association stored in the memory, for example, in order to identify an electronic record of the designated clinical encounter corresponding to the patient. The electronic record of the designated clinical encounter is modified to include the one or more clinical findings from the first clinical user for the designated clinical encounter corresponding to the patient from the incoming electronic communication, in various aspects.

In yet another aspect, a system is provide. The system includes a database that stores a mapping of a plurality of unique identifiers to a plurality of designated clinical encounters, in aspects. In some aspects, the system includes a computing device coupled to the database, where the computing device comprising a processor, a memory, a first service, and a second service, where the processor executes computer-executable instructions in the memory that operate the first service and the second service. The first service receives a request to transmit an outgoing electronic communication and a designated clinical encounter, and the designated clinical encounter includes one or more digitized images captured for a clinical encounter of a patient, in one aspect. The first service, in various aspects, automatically generates the outgoing electronic communication in response to the request, where the outgoing electronic communication includes the designated clinical encounter for review by a first clinical user. The first service automatically transmits the outgoing electronic communication including the designated clinical encounter to the first clinical user, in aspects. The outgoing electronic communication has a unique identifier that is associated with the designated clinical encounter.

The second service in the system receives an indication of the request to transmit the outgoing electronic communication and the designated clinical encounter. In response to receiving the indication, the second generates the unique identifier for the designated clinical encounter, the unique identifier being free of Patient Identifying Information (PII). The second service assigns the unique identifier to the designated clinical encounter, in some aspects, and stores an association of the designated clinical encounter and the unique identifier assigned to the designated clinical encounter in the memory. In aspects, the second services receives a second indication of the outgoing electronic communication and the designated clinical encounter being transmitted. In response to the second indication, the second service monitors for an incoming electronic communication that includes the unique identifier assigned to the designated clinical encounter, in one aspect. Based on the monitoring, the second service receives the incoming electronic communication from the first clinical user that includes the unique identifier assigned to the designated clinical encounter in the memory, wherein the incoming electronic communication including one or more clinical findings from the first clinical user for the designated clinical encounter, in various aspects. In one aspect, the second service, extracts the unique identifier from the incoming electronic communication and maps the unique identifier to the designated clinical encounter based on the association stored in the memory, for example, in order to identify an electronic record of the designated clinical encounter corresponding to the patient. The second service modifies the electronic record of the designated clinical encounter to include the one or more clinical findings from the first clinical user for the designated clinical encounter corresponding to the patient from the incoming electronic communication, in aspects.

### BRIEF DESCRIPTION OF THE DRAWINGS

Aspects are described in detail below with reference to the attached drawings figures, wherein:

FIG. 1 is a block diagram of a system, in accordance with aspects of the present disclosure;

FIG. 2 depicts a flowchart of a method, in accordance with aspects of the present disclosure;

FIG. 3 depicts a flowchart of another method, in accordance with aspects of the present disclosure;

FIG. 4 depicts a graphical user interface listing a plurality of clinical encounters, in accordance with aspects of the present disclosure;

FIG. 5 depicts a pop-up menu for triggering an outgoing electronic communication that includes a designated clinical encounter selected in the graphical user interface of FIG. 4, in accordance with aspects of the present disclosure;

FIG. 6A depicts an electronic communication that includes a designated clinical encounter sent from a sender service to an external application, in accordance with aspects of the present disclosure;

FIG. 6B depicts a registration of the electronic communication that includes a designated clinical encounter sent from a sender service to an external application, in accordance with aspects of the present disclosure;

FIG. 7A depicts an electronic communication having one or more clinical findings sent from an external application, in accordance with aspects of the present disclosure;

FIG. 7B depicts a registration of the electronic communication having one or more clinical findings sent from an external application, in accordance with aspects of the present disclosure; and

FIG. 8 depicts a computing environment suitable for implements of the aspects of the present disclosure.

### DETAILED DESCRIPTION

The subject matter of the present invention is being described with specificity herein to meet statutory requirements. However, the description itself is not intended to limit the scope of this patent. Rather, the inventors have contemplated that the claimed subject matter might also be embodied in other ways, to include different steps or combinations of steps similar to the ones described in this document, in conjunction with other present or future technologies. Terms should not be interpreted as implying any particular order among or between various steps herein disclosed unless and except when the order of individual steps is explicitly described. As such, although the terms “step” and/or “block” can be used herein to connote different elements of system and/or methods, the terms should not be interpreted as implying any particular order and/or dependencies among or between various components and/or steps herein disclosed unless and except when the order of individual steps is explicitly described. The present disclosure will now be described more fully herein with reference to the accompanying drawings, which may not be drawn to scale and which are not to be construed as limiting. Indeed, the present invention can be embodied in many different forms and should not be construed as limited to the aspects set forth herein. Further, it will be apparent from this Detailed Description that the technological solutions disclosed herein are only a portion of those provided by the present invention. As such, the technological problems, solutions, advances, and improvements expressly referenced and explained herein should not be construed in a way that would limit the benefits, improvements, and/or practical application of the discussed aspects of the present invention.

Aspects herein provide methods, systems, and computer-readable media that provide one or more services in a centralized and secure computerized healthcare records system. At a high level, the services, which include at least a sender service and a receiver service, automatically generate and transmit mobile-viewing-friendly electronic communications with seamlessly integrated identification-scrubbed clinical images, all while requiring minimal user input, such as a single user selection of a menu option for a selected record from a graphical user interface. Further, the services can perform these operations without requiring a user to navigate to a new window and/or to install specific applications. The services automatically transmit these image-laden electronic communications from the centralized and secure computerized healthcare records system to an external application that is accessible on a mobile device by a remote user, for example, who as the intended recipient is prompted to review the identification-scrubbed clinical images on the mobile device and reply with clinical findings. Because the electronic communications are scrubbed of patient identifying information (PII), the services utilize unique identifiers to track the electronic communications for individual clinical encounters. Additionally, the services automatically track replies as incoming electronic communications that can be mapped back to one patient and one patient visit using the unique identifiers, in aspects. As each incoming communication is received and mapped, the services automatically extract clinical findings from the incoming communication and provide a notification of the clinical findings to the appropriate clinician for the patient that corresponds to the reviewed images, within the secure computerized healthcare records system. The unique identifiers are purged after clinical findings are extracted from the incoming electronic communications, and as such, the unique identifiers are not used more than once for any single patient to ensure patient information is not shared outside the secure healthcare system.

As used herein, the terms “first,” “second,” “third,” and so forth are used to merely distinguish between features for the purpose of a clear description. These terms are not indicators of relative importance and should not be interpreted as implying any particular order among or between various features herein disclosed unless and except when a sequence is explicitly described. The terms “operation” and “function” are used interchangeably to refer to computer-executable instructions that are used to specify the performance of actions, tasks, queries, searches, record returns, and the like, for example, by a computing device such as a server.

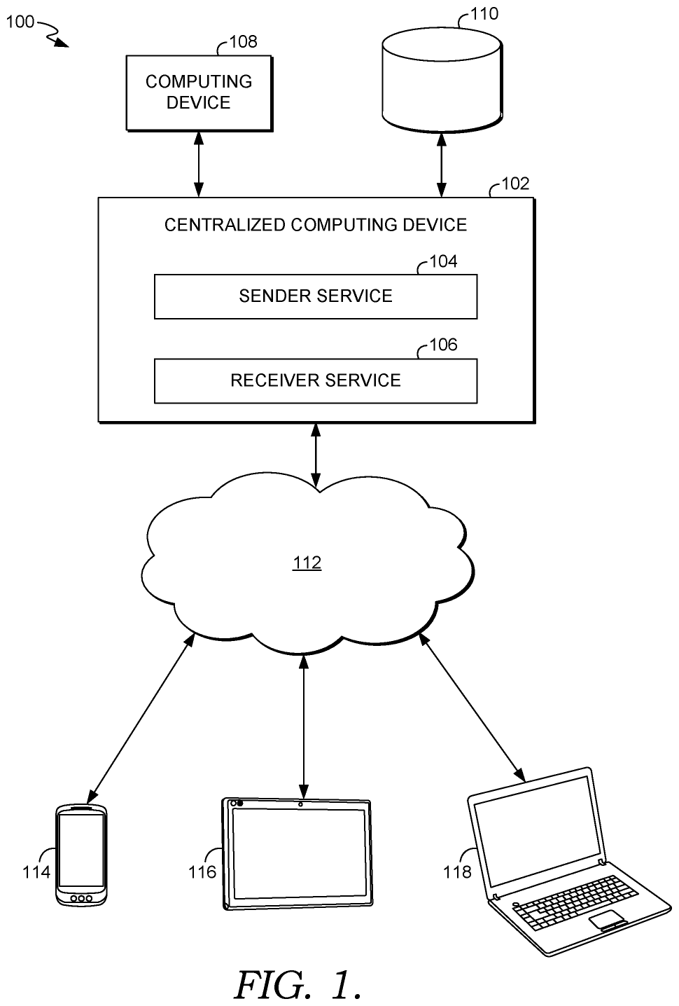

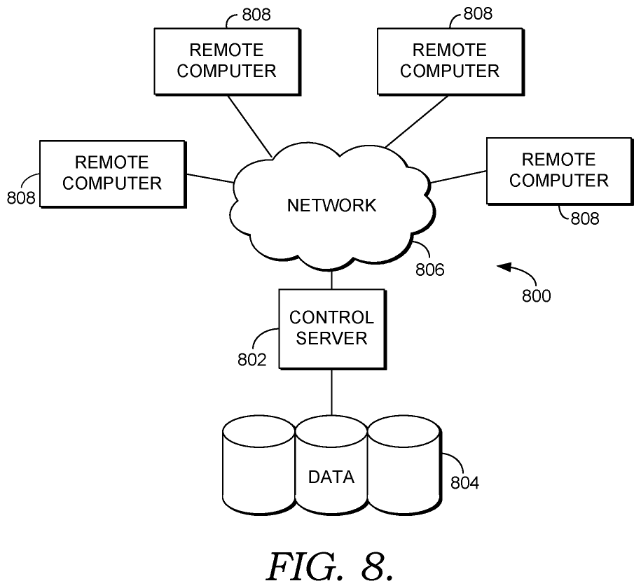

Beginning with FIG. 1, a system environment 100 is provided. The system environment 100 can include a centralized computing device 102, a sender service 104, a receiver service 106, an internal computing device 108, a database 110, a network 112, and one or more external computing devices, such as a mobile telephone 114, a tablet device 116, and a laptop computing device 118, for example. In aspects, the centralized computing device 102 is a computing device having a processor and a memory, wherein the processor executes computer-executable instructions in the memory. For example, the centralized computing device 102 can be a physical server, a virtual server, a laptop computer, a desktop computer, a cart-type medical device, a workstation computing device, or any other type of computing device as described with reference to FIG. 8.

The centralized computing device 102 is “centralized,” meaning that the device is integrated into a secure computerized healthcare records system, either by direct hardwired connections and/or through wireless communications. The centralized computing device 102 is communicatively coupled to the network 112 of the system environment 100, which may be internal as controlled and hosted by the secure computerized healthcare records system, or which may be a non-secure network that is external to the secure computerized healthcare records system. For example, the network 112 may be a Wi-Fi network, a mesh network, an ad-hoc network, a telecommunications network, a peer-to-peer network, and/or the network types described in FIG. 8. In embodiments, the centralized computing device 102 hosts a system that includes the sender service 104 and the receiver service 106. In an embodiment, the centralized computing device 102 can include a processor and a memory, wherein the processor executes computer-executable instructions in the memory that operate the sender service 104 and the receiver service 106. In various examples herein, the sender service 104 may be interchangeably referred to as a first service and the receiver service 106 may be interchangeably referred to as a second service.

At a high level, the sender service 104 operates to automatically generate and transmit Patient Identifying Information (PII)-scrubbed electronic communications, such as emails or text messages, to an electronic address that corresponds to a clinician. The electronic communications may include one or more digitized images that are attached to and/or are integrated with the electronic communication, or alternatively, that may be accessed through a navigation link in the electronic communication to a secure web-based application, in various aspects. Generally, the one or more digitized images are provided so the clinician can remotely view high resolution images on any type or kind of mobile device, without requiring a separate application or tool be installed, and further, can provide clinical findings in response, through the receiver service 106. The digitized images may be clinical images captured as part of a patient's physical examination and/or clinical diagnostic testing, in some aspects. For example, the one or more digitized images include one or more of: a radiology image; a nuclear radiology image; a mammogram; a Computerized Axial Tomography (CAT) image; a Positron Emission Tomography (PET) image; a Magnetic Resonance Image (MRI); an x-ray image; an ultrasound image; a photograph; a video; a fluoroscopy image; or a pathology specimen image (e.g., stained slide having a sample, test tube containing bodily fluid, a stained biopsy sample).

The sender service 104 initially receives a request to transmit an outgoing electronic communication and a designated clinical encounter, the designated clinical encounter including one or more digitized images captured for a clinical encounter of a patient. The request or an indication of the request may be received by the sender service 104 from the internal computing device 108. For example, a user (e.g., an imaging technician) may input a request to have a first clinical user (e.g., a radiologist) review and provide clinical findings for a particular, selected, or otherwise designated clinical encounter where a set of radiology images were captured of a patient. The sender service 104 automatically generates an outgoing electronic communication in response to that request, in aspects, and the outgoing electronic communication includes the digitized images of the designated clinical encounter for review by a first clinical user. The sender service 104 may further automatically transmit the outgoing electronic communication that includes the designated clinical encounter to the first clinical user, in aspects. Generally, the outgoing electronic communication has a unique identifier that is associated with the designated clinical encounter, albeit the designed clinical encounter having the digitized images and the unique identifier have automatically been scrubbed free of any and all PII.

In various aspects, the sender service 104 may (i) automatically generate the electronic communication that includes one or more digitized images of the designated clinical encounter, including automatic lookup of a first clinical user identified for reviewing the designated clinical encounter based on a medical order and retrieval of an electronic address for the first clinical user, (ii) automatically scrub Patient Identifying Information (PII) from the designated clinical encounter/one or more digitized images prior to transmitting the outgoing electronic communication, and (iii) automatically transmit the electronic communication that includes one or more digitized images to the first clinical user using minimal user interactions. For example, the sender service 104 may perform all of the actions above without requiring a user to navigate to an email application in the secure computerized healthcare records system, and/or by using a single user interaction to trigger or instruct the sender service from a menu (e.g., one click selection of an option from menu in a graphical user interface).

The sender service 104 transmits the electronic communication as “outgoing” from the secure computerized healthcare records system to an external application. Generally, the external application (e.g., a web-based email application, a messaging application) is accessible on a mobile device by the first clinical user. As such, the external application provides the first clinical user access to their electronic address (e.g., email address, phone number, a messaging application) via a mobile device, such as a mobile telephone 114, a tablet device 116, and/or a laptop computing device 118, in aspects. As such, the first clinical user can view the one or more digitized images from the clinical encounter directly on the mobile device, as the one or more digitized images have been integrated with the electronic communication sent by the sender service 104.

Additionally or alternatively, the sender service 104 may generate a selectable link that, when selected, automatically directs the first clinical user to a secure web-based high-resolution image viewer for entry of credentials to login to the secure web-based high-resolution image viewer, in such aspects. In one such aspect, the secure web-based high-resolution image viewer enables display, subsequent to successful verification of the credentials entered, of one or more digitized images that correspond to the designated clinical encounter via a mobile device. As such, the sender service 104 may, in some aspects, integrate the selectable link into the outgoing electronic communication, wherein the selectable link is integrated by attachment to the outgoing electronic communication or by insertion into a message body of the outgoing electronic communication.

The centralized computing device 102 also includes a receiver service 106, which works in tandem with or concurrently with the sender service 104. In aspects, the receiver service 106 receives an indication of the request to transmit the outgoing electronic communication and the designated clinical encounter. The indication may be sent by the sender service 104 and/or another service within the secure computerized healthcare records system, in various aspects. In response to receiving the indication, the receiver service 106 generates a unique identifier for the designated clinical encounter, the unique identifier being free of Patient Identifying Information (PII). The receiver service 106 provides this unique identifier to the sender service 104 for inclusion with the outgoing electronic communication. Alternatively, the receiver service 106 may generate and provides the PII-free unique identifier to the sender service 104 in response to the sender service 104 automatically generating the outgoing email, as discussed above.

The receiver service 106 assigns the unique identifier to the designated clinical encounter, in aspects, and receiver service 106 stores an association of the designated clinical encounter and the unique identifier assigned to the designated clinical encounter in the memory of the database 110, in various aspects. Accordingly, the receiver service 106 of the centralized computing device 102 is communicatively coupled to a database 110. In aspects, the receiver service 106 can use the stored association of the unique identifier to individually track incoming and outgoing electronic communications that are specific to one particular clinical encounter, in some aspects. The database 110 may include one or more types of virtual and/or physical memory, and may include any and all of the features of the database described with reference to FIG. 8.

The receiver service 106 may, in some aspects, receive a second indication of the outgoing electronic communication and the designated clinical encounter as being transmitted from the sender service 104 to an electronic address that is specific to one particular first clinical user who is prompted by the outgoing electronic communication to review the clinical encounter (e.g., including one or more digitized images). In response to the second indication, the receiver service 106 may perform periodic or continuous monitoring of incoming electronic communications to determine which, if any, include the unique identifier assigned to the designated clinical encounter. Based on the monitoring, the receiver service 106 receives an incoming electronic communication from the first clinical user that includes the unique identifier assigned to the designated clinical encounter as stored in the memory of the database 110, in aspects. The incoming electronic communication can include, generally, one or more clinical findings input by the first clinical user that relate to the designated clinical encounter.

Accordingly, the receiver service 106 extracts the unique identifier from the incoming electronic communication, in some aspects. Using the unique identifier extracted from the incoming electronic communication, the receiver service 106 maps the unique identifier to the designated clinical encounter based on the association stored in the memory, in aspects, in order to identify an electronic record of the designated clinical encounter corresponding to the patient. In some aspects, the receiver services matches the unique identifier extracted from the incoming electronic communication to the unique identifier stored in the database, and identifies the designated clinical encounter that is associated with unique identifier as stored in the database. In one an aspect, the designated clinical encounter that is identified can be used by the receiver service 106 to search and locate the associated records (e.g., a medical order that was entered to order the designated clinical encounter, a contemporaneous record or documentation of the designated clinical encounter by a technician obtaining images of the patient) within the secure computerized healthcare records system and to identify an electronic medical record (EMR) and/or an electronic health record (HER) for the patient who corresponds to the clinical encounter.

The receiver service 106 can, in some aspects, modify an electronic record of the designated clinical encounter to include the one or more clinical findings from the first clinical user for the designated clinical encounter corresponding to the patient from the incoming electronic communication. For example, the receiver service 106 can extract the one or more clinical findings (e.g., text data or voice data) that were integrated in the incoming electronic communication received, and the one or more clinical findings can be inserted into the electronic record of the designed clinical encounter that is maintained within the secure computerized healthcare records system. Additionally or alternatively, the receiver service 106 can extract the one or more clinical findings (e.g., text data or voice data) that were integrated in the incoming electronic communication received, and the one or more clinical findings can be inserted into the electronic record of a specific patient the corresponds to the designed clinical encounter, where the patient's electronic record is maintained within the secure computerized healthcare records system.

In response to and/or subsequent to modifying the electronic record of the designated clinical encounter and/or patient to include the one or more clinical findings, the receiver service 106 deletes the association of the designated clinical encounter and the unique identifier from the memory, in further aspects. This deletion ensures the unique identifier is never re-utilized in such a way that the unique identifier could be used to identify a specific patient, for example.

The receiver service 106 may identify a second clinical user that is associated with the designated clinical encounter corresponding to the patient and an electronic address of the second clinical user, in aspects. For example, the second clinical user may be a clinician that initially placed a medical order for the designated clinical encounter of the patent, or may be a primary care clinician for the patient. The receiver service 106 may generate a notification to be communicated to the electronic address of the second clinical user, wherein that notification includes the one or more clinical findings from the first clinical user for the designated clinical encounter corresponding to the patient, in some aspects. In such aspects, the receiver service 106 and/or the sender service 104 can communicate the notification to the second clinical user. The second clinical user can then access, read, and review the one or more clinical findings provided by the first clinical user.

Having described the system environment 100 and components thereof, it will be understood by those of ordinary skill in the art that system environment 100 is but one example of a suitable system and is not intended to limit the scope of use or functionality of the present invention. Similarly, system environment 100 should not be interpreted as imputing any dependency and/or any requirements with regard to each component and combination(s) of components illustrated in FIG. 1. It will be appreciated by those of ordinary skill in the art that the location of components illustrated in FIG. 1 is an example, as other methods, hardware, software, components, and devices for establishing a communication links between the components shown in FIG. 1, may be utilized in implementations of the present invention. It will be understood to those of ordinary skill in the art that the components may be connected in various manners, hardwired or wireless, and may use intermediary components that have been omitted or not included in FIG. 1 for simplicity's sake. As such, the absence of components from FIG. 1 should be not be interpreted as limiting the present invention to exclude additional components and combination(s) of components. Moreover, though components are represented in FIG. 1 as singular components, it will be appreciated that some embodiments may include a plurality of devices and/or components such that FIG. 1 should not be considered as limiting the number of a device or component.

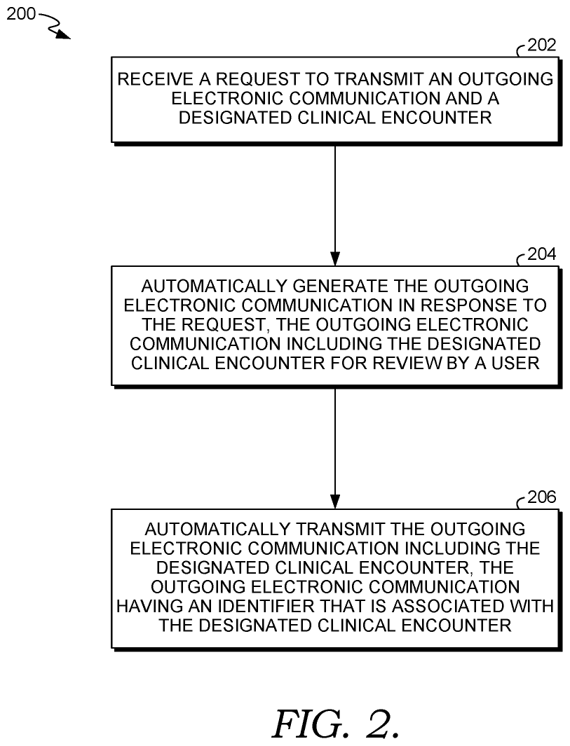

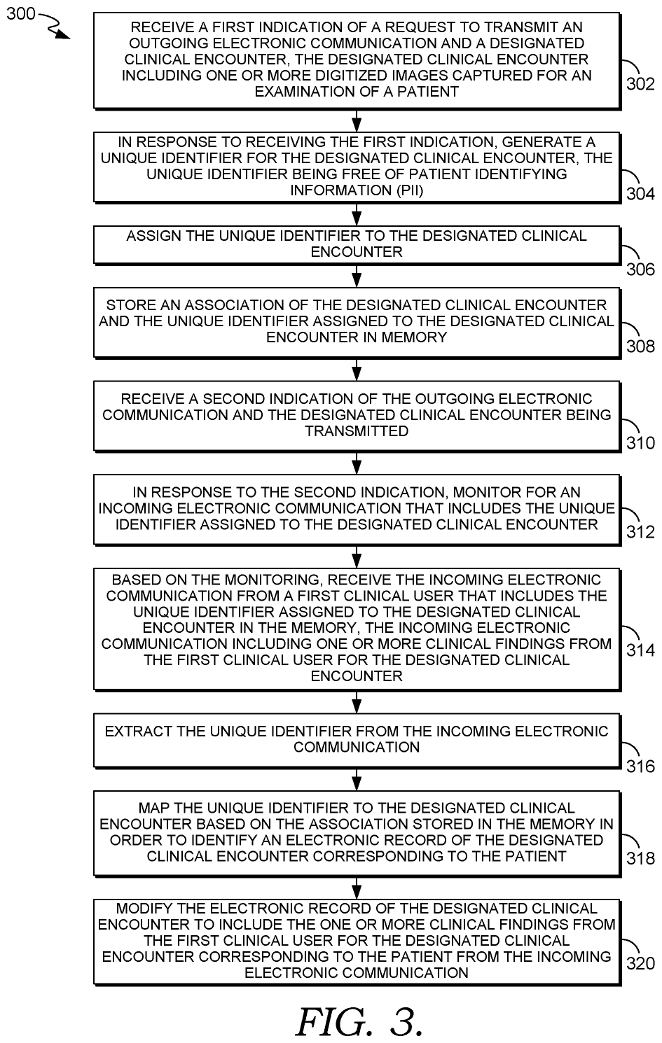

Turning now to FIGS. 2 and 3, methods are discussed that can be performed via one or more of the components and component interactions previously described in FIG. 1. As such, the methods are discussed briefly for brevity, though it will be understood that the previous discussion and details described therein can be applicable to aspect of the methods of FIGS. 2 and 3. Additionally or alternatively, it will be understood that the methods discussed herein can be implemented or performed via the execution of computer-readable instructions stored on computer-readable media, by one or more processors. For example, each of FIGS. 2 and 3 may provide methods that are performed when one more non-transitory computer-readable media having computer-executable instructions embodied thereon are executed by one or more processors.

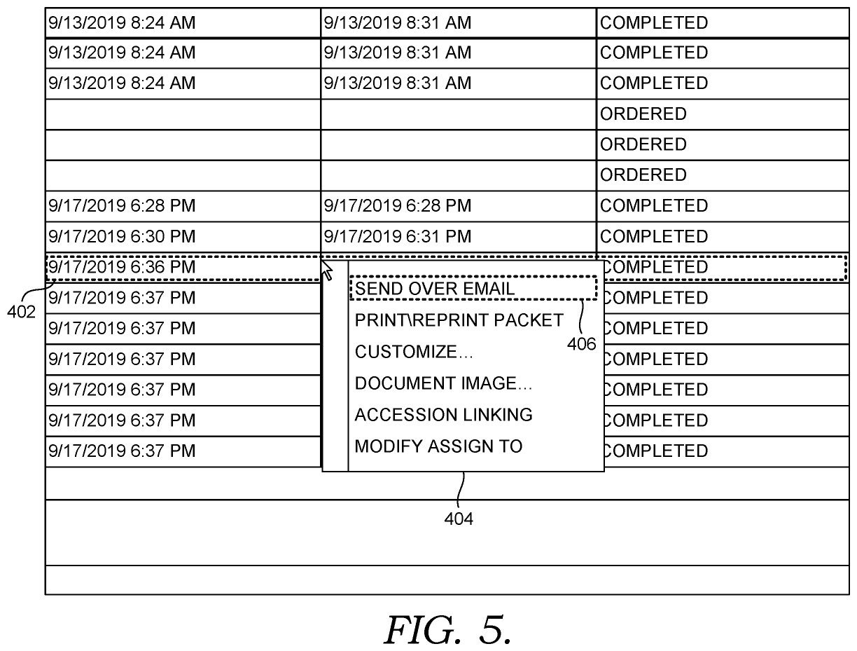

FIG. 2 provides a method 200 for the sender service 104 of FIG. 1. At block 202, a request to transmit an outgoing electronic communication and a designated clinical encounter is received. In various aspects, the electronic communication is deemed to be “outgoing” as being sent from the internal system, such as the centralized computing device 102 operating in a secure computerized healthcare records system, to an external application that is accessible on one or more mobile devices, such as the mobile telephone 114, for example, shown in FIG. 1. In one example, a graphical user interface 400 of FIG. 1 may present a plurality of selectable rows, each row corresponding to one clinical encounter such as patient examination where one or more images were captured of the patient and/or a sample or specimen of the patient. One or more columns may be presented in the graphical user interface 400 to present related information for each clinical encounter having a row. In the detail of the graphical user interface 400 shown in FIG. 5, a user may use a pointer or cursor to right-click on row 402, which both selects and designates one of the clinical encounters (row is shown with a dashed line) and at the same time causes a pop-up menu 404 to be displayed as overlaid the graphical user interface 400. With one click of the “SEND OVER EMAIL” option 406 from the pop-up menu 404, the sender service 104 receives this selection as a request to transmit an outgoing electronic communication with the designated clinical encounter (e.g., including one or more digitized images) of the selected row. The sender service 104 can proceed with the method 200 without further user interaction. As such, the request received can include the selection of one particular clinical encounter, and that one clinical encounter becomes the designated clinical encounter for the method 300 discussed, based on the selection in the request.

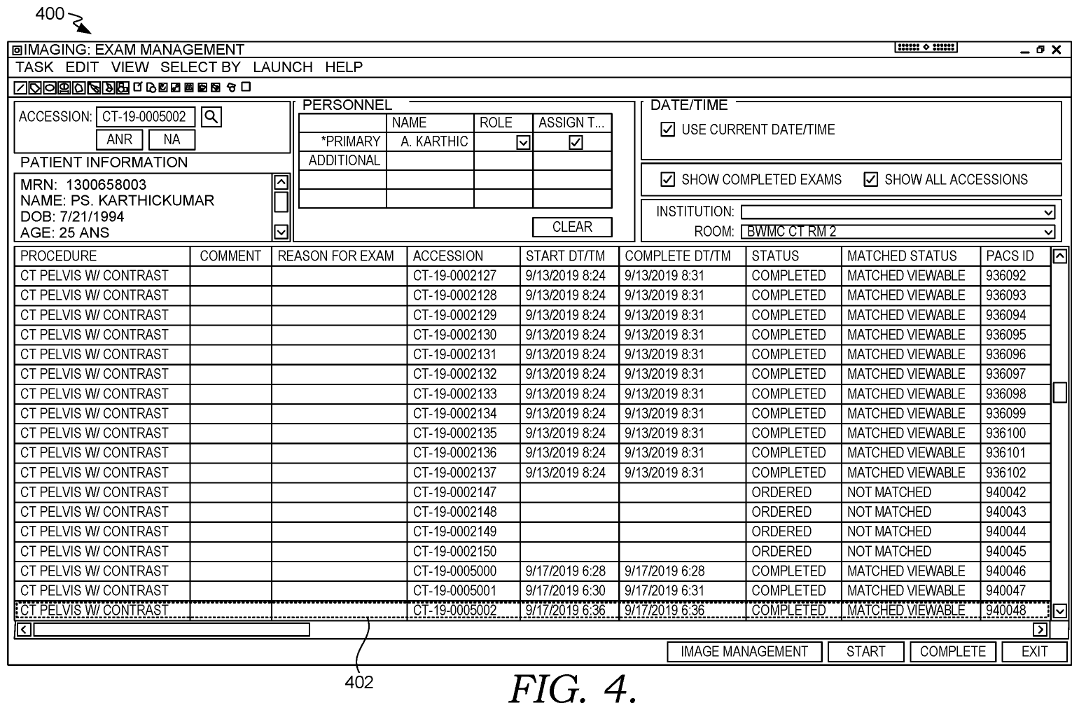

At block 204, the outgoing electronic communication is automatically generated in response to the request, where the outgoing electronic communication includes the designated clinical encounter for review by a user. In some aspects, automatically generating the outgoing electronic communication includes retrieving an electronic record of the designated clinical encounter. For example, the electronic record of the designated clinical encounter can include one or more digitized images (e.g., a plurality of digitized CT scans from the “CT PELVIS W/ CONTRAST” clinical encounter identified as CT-19-0005002, and designated as selected in FIGS. 4 and 5) that are retrieved by the sender service 104. The sender service 104 can integrate the one or more digitized images with the outgoing electronic communication, in various aspects. For example, the one or more digitized images may be “integrated” by attachment to the outgoing electronic communication and/or by insertion into a message body of the outgoing electronic communication.

The sender service can, prior to transmitting the outgoing electronic communication, automatically scrub Patient Identifying Information (PII) from the designated clinical encounter, including text and/or metadata having PII for one or more digitized images. Additionally, the sender service 104 can integrate a unique identifier in the outgoing electronic communication, wherein the unique identifier specifically corresponds to the designated clinical encounter and does not include PII. The unique identifier may be received by the sender service 104 from the receiver service 106, as discussed herein.

In some aspects, automatically generating the outgoing electronic communication includes identifying the user who is associated with the designated clinical encounter, where the user is a clinician, for example. The sender service 104 may also identify an electronic address that is assigned to the user identified as being associated with the designated clinical encounter, in such aspects. Additionally, the sender service 104 may automatically generate the outgoing electronic communication by identifying the user who is associated with a patient corresponding to the designated clinical encounter, wherein the user is a clinician who can be identified by the sender service 104, for example, from a medical order requesting the designated clinical encounter of the patient. The sender service 104 may identify an electronic address that is assigned to the user who has been identified as being associated with the patient corresponding to the designated clinical encounter, in such aspects. In one example, the user is a first clinical user whose clinical interpretation of digitized images of the designated clinical encounter is being requested. Accordingly, the sender service 104 can automatically generate the outgoing electronic communication using the electronic address identified for the user as the intended recipient. The electronic address may be identified from within the medical order for the designated clinical encounter that can specify an intended clinician to review digitized images of the designated clinical encounter, in some aspects. In this manner, a user requesting the outgoing electronic communication from a graphical user interface as shown in FIGS. 4 and 5 does not need to open a new application, type in clinical information, search for an electronic address (email), find and attach images for the designated clinical encounter, add text, or the like.

Additionally or alternatively, automatically generating the outgoing electronic communication includes generating a selectable link that, when selected, automatically directs the user to a secure web-based high-resolution image viewer for entry of credentials to login to the secure web-based high-resolution image viewer, in some aspects. The secure web-based high-resolution image viewer enables display, subsequent to successful verification of the credentials entered, of one or more digitized images that correspond to the designated clinical encounter, for example. In such an aspect, the sender service 104 may automatically integrate the selectable link with the outgoing electronic communication, wherein the selectable link is integrated by attachment to the outgoing electronic communication or by insertion into a message body of the outgoing electronic communication, for example.

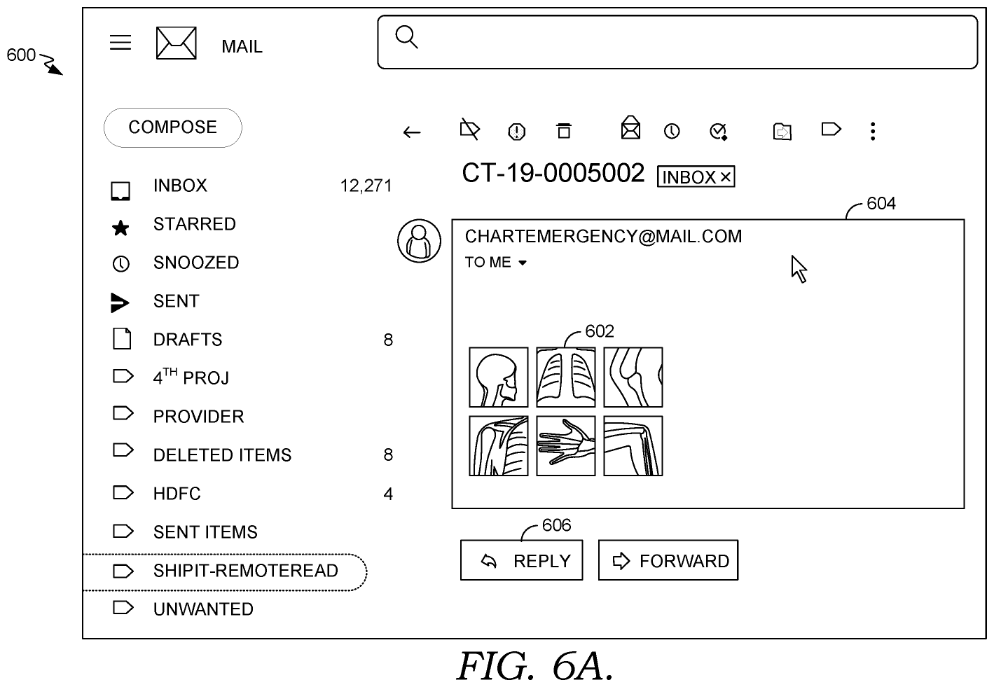

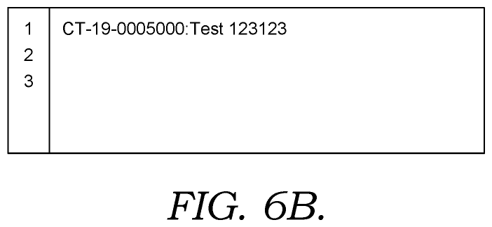

At block 206, the outgoing electronic communication that includes the designated clinical encounter is automatically transmitted, where outgoing electronic communication has an identifier that is associated with the designated clinical encounter. For example, the graphical user interface 600 of FIG. 6A includes an external application (e.g., web-based email), that is accessible on a mobile device by a user intended to review the designated clinical encounter. As such, the external application provides the user access to their electronic address (e.g., email address, phone number, a messaging application) via a mobile device, such as a mobile telephone 114, a tablet device 116, and/or a laptop computing device 118, in aspects. Further, the user can immediately access and view the one or more digitized images 602, as shown in FIG. 6A integrated with the electronic communication 604. As such, and as further described below, the user can review the images on a mobile device through the external application, and can select a reply button 606 to draft a responsive electronic communication having one or more clinical findings from the image review, for example.

Continuing, FIG. 3 provides a method 300 for the receiver service 106 of FIG. 1. At block 302, for each one of a plurality of designated clinical encounters, a first indication of a request to transmit an outgoing electronic communication and a designated clinical encounter is received, where the designated clinical encounter includes one or more digitized images captured for a clinical encounter of a patient. The receiver service 106, as mentioned hereinabove, may receive the indication directly or indirectly from the sender service 104, for example, in response to the request for an outgoing electronic communication, or in response to the automatic generation of the outgoing electronic communication.

At block 304, in response to receiving the first indication, a unique identifier is generated for the designated clinical encounter, where the unique identifier is free of any and all Patient Identifying Information (PII). The unique identifier may be a string of a plurality of alphanumeric characters and/or non-alphanumeric characters. In some aspects, the unique identifier is randomly generated by the receiver service to ensure the designated clinical encounter and the outgoing electronic communication being generated by the sender service cannot be used to reverse identify a particular patient.

At block 306, the unique identifier is assigned to the designated clinical encounter. At block 308, an association of the designated clinical encounter and the unique identifier assigned to the designated clinical encounter is stored in memory. For example, as shown in FIG. 6B, the outgoing electronic communication having the unique identifier shown as “CT-19-0005002” can be stored and logged in the database 110 for the designated clinical encounter “Test 123.” Although an accession number is used throughout the figures for simplicity and illustration, it will be understood that a randomized or non-accession identifier is generated and utilized. At block 310, a second indication is received that the outgoing electronic communication and the designated clinical encounter is being transmitted. For example, the sender service 104 transmits the outgoing electronic communication and the designated clinical encounter out of the secure computerized healthcare records system to the electronic address of an intended recipient, such as a first clinical user.

In some aspects, the receiver service 106 performs these functions for a plurality of unique identifiers which are then generated and assigned to a plurality of designated clinical encounters. As such, a plurality of associations are stored for unique identifiers as they correspond to designated clinical encounters in the memory.

At block 312, incoming electronic communications are monitored for the unique identifier assigned to the designated clinical encounter, in response to the second indication. In various aspects, an electronic communication is deemed to be “incoming” as being received by the internal system (e.g., a secure computerized healthcare records system), and as being sent from an external application, for example. In various aspects, the receiver service 106 receives a plurality of incoming electronic communications, scans each of the plurality of incoming electronic communications for the unique identifier, and recognizes when one of the plurality of incoming electronic communications is the incoming electronic communication that includes the unique identifier assigned to the designated clinical encounter in the memory.

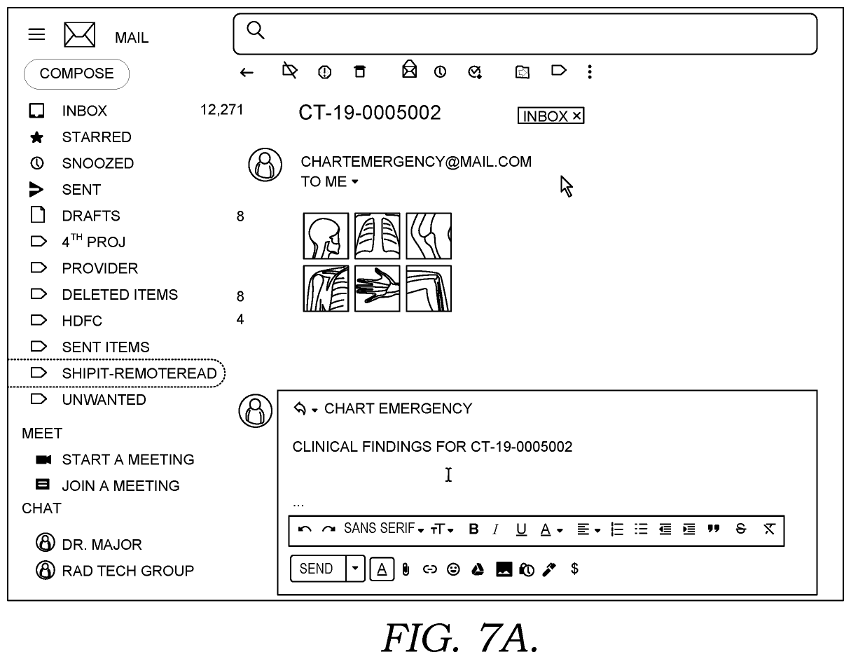

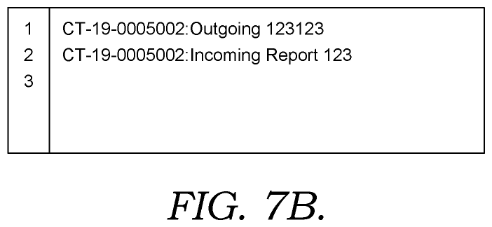

At block 314, based on the monitoring, an incoming electronic communication is received from a first clinical user which includes the unique identifier assigned to the designated clinical encounter in the memory, wherein the incoming electronic communication includes one or more clinical findings from the first clinical user for the designated clinical encounter. As shown in FIG. 7A, the incoming electronic communication presented in the external application includes the unique identifier “CT-19-0005002.” The body of the incoming electronic communication may include one or more clinical findings, for example, that are responsive to the first clinical user's review of the one or more digitized images that correspond to the designed clinical encounter. In some aspects, the incoming electronic communication is an externally-generated email. In other aspects, the incoming electronic communication is received from a secure web-based high-resolution image viewer.

At block 316, the unique identifier is extracted from the incoming electronic communication. At block 318, the unique identifier is mapped to the designated clinical encounter based on the association stored in the memory in order to identify an electronic record of the designated clinical encounter corresponding to the patient. For example, as shown in FIG. 7B, the incoming electronic communication having the unique identifier shown as “CT-19-0005002” can be stored and logged in the database 110 for the designated clinical encounter “Test 123.” In some aspects, mapping the unique identifier to the designated clinical encounter based on the association stored in the memory includes searching the memory for the unique identifier, identifying that the unique identifier is assigned to the designated clinical encounter in the memory, and locating the electronic record that corresponds to the designated clinical encounter.

At block 320, the electronic record of the designated clinical encounter is modified to include the one or more clinical findings from the first clinical user for the designated clinical encounter corresponding to the patient from the incoming electronic communication. Subsequent to modifying the electronic record of the designated clinical encounter to include the one or more clinical findings, the receiver service 106 deletes or purges the association of the designated clinical encounter and the unique identifier from the memory. In some aspects, the receiver service 106 individually deletes each association of a plurality of unique identifiers with a plurality of designated clinical encounters when, for example, each of the plurality of unique identifiers in the plurality of incoming electronic communications are individually mapped, for example.

In further embodiments, the receiver service 106 and/or the sender service 104 can provide one or more notifications to clinical users that report the receipt of the one or more clinical findings extracted from the incoming electronic communication that is received from the first clinical user (e.g., a radiologist). In such aspects, a second clinical user (e.g., a primary care physician) who is associated with the designated clinical encounter corresponding to the patient is identified, and an electronic address of the second clinical user is identified. For example, the second clinical user may be a clinician who placed a medical order for the designated clinical encounter of that patient. The receiver service 106 and/or the sender service 104 can generate a notification to be communicated to the electronic address of the second clinical user, wherein the notification includes the one or more clinical findings from the first clinical user for the designated clinical encounter corresponding to the patient. Then, the notification is transmitted to the second clinical user, for example, within the secure computerized healthcare records system, and/or to mobile device or external application.

Additionally, the operations, functions, and/or steps discussed above can correspond to a computer-implemented method. In one aspect, one or more non-transitory computer-readable media having computer-readable instructions or computer-readable program code portions embodied thereon, for execution via one or more processors, can be used to implement and/or perform the operations, functions, and/or steps discussed. For example, computer-readable instructions or computer-readable program code portions can specify a sequence of operations, functions, and/or steps, and/or can identify particular component(s) of a software and/or hardware for performing one or more operations, functions, and/or steps. The computer-readable instructions or computer-readable program code portions can correspond to an application and/or an application programming interface (API), in some aspects. Accordingly, as discussed, the operations, functions, and/or steps can be performed using software, hardware, component(s), and/or device(s) depicted in the example of FIG. 1.

Turning to FIG. 8, a computing environment 800 that is suitable for use in implementing aspects of the present invention is depicted. The computing environment 800 is merely an example of one suitable computing environment and is not intended to suggest any limitation as to the scope of use or functionality of the invention. The computing environment 800 should also not be interpreted as having any dependency or requirement relating to any single component or combination of components illustrated therein. Generally, in aspects, the computing environment 800 is a medical-information computing-system environment.

However, this is just one example and the computing environment 800 can be operational with other types, other kinds, or other purpose computing system environments or configurations. Examples of computing systems, environments, and/or configurations that might be suitable for use with the present invention include personal computers, server computers, hand-held or laptop devices, multiprocessor systems, microprocessor-based systems, set top boxes, programmable consumer electronics, network PCs, minicomputers, mainframe computers, distributed computing environments that include any of the above-mentioned systems or devices, and the like.

In aspects, the computing environment 800 can be described in the general context of computer instructions, such as program modules, applications, and/or extensions, being read and executed by a computing device. Examples of computer instructions can include routines, programs, objects, components, and/or data structures that perform particular tasks or implement particular abstract data types. The aspects discussed herein can be practiced in centralized and/or distributed computing environments, i.e., where computer tasks are performed utilizing remote processing devices that are linked through a communications network, whether hardwired, wireless, or a combination thereof. In a distributed configuration, computer instructions might be stored or located in association with one or more local and/or remote computer storage media (e.g., memory storage devices). Accordingly, different portions of computer instructions for implementing the computer tool in the computing environment 800 may be executed and run on different devices, whether local, remote, stationary, and/or mobile.

With continued reference to FIG. 8, the computing environment 800 comprises a computing device 802, shown in the example form of a server. Although illustrated as one component in FIG. 8, the present invention can utilize a plurality of local servers and/or remote servers in the computing environment 800. The computing device 802 can include components such as a processing unit, internal system memory, and a suitable system bus for coupling to various components, including electronic storage, memory, and the like, such as a data store, a database, and/or a database cluster. Example components of the computing device 802 include a processing unit, internal system memory, and a suitable system bus for coupling various components, including a database 804, with the computing device 802. An example system bus might be any of several types of bus structures, including a memory bus or memory controller, a peripheral bus, and a local bus, using any of a variety of bus architectures. Examples of bus architectures include Industry Standard Architecture (ISA) bus, Micro Channel Architecture (MCA) bus, Enhanced ISA (EISA) bus, Video Electronics Standards Association (VESA®) local bus, and Peripheral Component Interconnect (PCI) bus, also known as Mezzanine bus.

The computing device 802 includes or has access to a variety of non-transitory computer-readable media. Computer-readable media can be any available media that is locally and/or remotely accessible by the computing device 802, and includes volatile, nonvolatile, removable, and non-removable media. By way of example, and not limitation, computer-readable media can comprise computer storage media and communication media. Computer storage media includes volatile, nonvolatile, removable, and non-removable media, as implemented in any method or technology for storage of information, such as computer-readable instructions, data structures, program modules, or other data.

The computing device 802 can include or can have access to computer-readable media. Computer-readable media can be any available media that can be accessed by computing device 802, and includes volatile and nonvolatile media, as well as removable and non-removable media. By way of example, and not limitation, computer-readable media can include computer storage media and communication media.

Computer storage media can include, without limitation, volatile and nonvolatile media, as well as removable and non-removable media, implemented in any method or technology for storage of information, such as computer-readable instructions, data structures, program modules, or other data. In this regard, computer storage media can include, but is not limited to, Random Access Memory (RAM), Read-Only Memory (ROM), Electrically Erasable Programmable Read-Only Memory (EEPROM), flash memory or other memory technology, CD-ROM, digital versatile disks (DVDs) or other optical disk storage, magnetic cassettes, magnetic tape, magnetic disk storage, or other magnetic storage device, or any other medium which can be used to store the desired information and which can be accessed by the computing device 802. Computer storage media does not encompass signal per se.

Communication media typically embodies computer-readable instructions, data structures, program modules, or other data in a modulated data signal, such as a carrier wave or other transport mechanism, and can include any information delivery media. As used herein, the term “modulated data signal” refers to a signal that has one or more of its attributes set or changed in such a manner as to encode information in the signal. By way of example, and not limitation, communication media includes wired media, such as a wired network or direct-wired connection, and wireless media, such as acoustic, radio frequency (RF), infrared, and other wireless media. Combinations of any of the above also can be included within the scope of computer-readable media.

The computing device 802 might operate in a network 806 using logical connections to one or more remote computers 808. In some aspects, the one or more remote computers 808 can be located at a variety of locations, such as medical facilities, research environments, and/or clinical laboratories (e.g., molecular diagnostic laboratories), as well as hospitals, other inpatient settings (e.g., surgical centers), veterinary environments, ambulatory settings, medical billing offices, financial offices, hospital administration settings, home healthcare environments, clinics, and/or clinical offices. As used herein, “clinicians,” “medical professionals,” or “healthcare providers” can include: physicians; specialists, such as surgeons, radiologists, cardiologists, and oncologists; emergency medical technicians; physicians' assistants; nurse practitioners; health coaches; nurses; nurses' aides; pharmacists; dieticians; microbiologists; laboratory experts; laboratory technologists; genetic counselors; researchers; veterinarians; students; and the like.

In aspects, the computing device 802 uses logical connections to communicate with one or more remote computers 808 within the computing environment 800. In aspects where the network 806 includes a wireless network, the computing device 802 can employ a modem to establish communications with the Internet, the computing device 802 can connect to the Internet using Wi-Fi or wireless access points, or the server can use a wireless network adapter to access the Internet. The computing device 802 engages in two-way communication with any or all of the components and devices illustrated in FIG. 8, using the network 806. Accordingly, the computing device 802 can send data to and receive data from the remote computers 808 over the network 806.

The network 806 is a computer network that can include local area networks (LANs) and/or wide area networks (WANs), in some aspects. The network 806 can include wireless and/or physical (e.g., hardwired) connections. Examples of networks include a telecommunications network of a service provider or carrier (e.g., 5G, LTE), Wide Area Network (WAN), a Local Area Network (LAN), a Wireless Local Area Network (WLAN), a cellular telecommunications network, a Wi-Fi network, a short range wireless network, a Wireless Metropolitan Area Network (WMAN), a Bluetooth® capable network, a fiber optic network, or a combination thereof. When the network 806 includes a WAN-type configuration, the computing device 802 might comprise a modem or other means for establishing communications over the WAN, such as the Internet, in such aspects. As such, the network 806, can provide the components and devices access to the Internet and web-based applications.

The network 806 can include an entity-wide network, campus-wide network, an office-wide network, an enterprise-wide networks, and the Internet. In the network 806, applications, extensions, program modules or portions thereof might be stored in association with the computing device 802, the database 804, and any of the one or more remote computers 808. For example, various application programs can reside on the memory associated with any one or more of the remote computers 808. In the computing environment 800, which is illustrated as being a distributed configuration of the network 806, the components and devices can communicate with one another and can be linked to each other using a network 806. It will be appreciated by those of ordinary skill in the art that the network connections shown are exemplary and other means of establishing a communications link between the computers (e.g., computing device 802 and remote computers 808) might be utilized.

In operation, an organization might enter commands and information into the computing device 802 or convey the commands and information, for example, directly in peer-to-peer or near-field communication, or through the network 806 using telecommunications or Wi-Fi, to the computing device 802 via one or more of the remote computers 808 through input devices, such as a keyboard, a pointing device (e.g., a mouse), a trackball, as stylus, or a touch pad. Other input devices comprise microphones, satellite dishes, scanners, or the like. Commands and information might also be sent directly from a remote healthcare device to the computing device 802. In addition to a screen, monitor, or touchscreen component, the computing device 802 and/or remote computers 808 might comprise other peripheral output devices, such as speakers and printers.

The computing environment 800 includes one or more remote computers 808, which may be accessed by the computing device 802 over the network 806 or directly using peer-to-peer connections or mesh networking, in various aspects. The remote computers 808 might be servers, routers, network personal computers, peer devices, network nodes, computing devices, personal digital assistants, personal mobile devices, medical devices, patient monitoring equipment, or the like, and might comprise some or all of the elements described above in relation to the computing device 802. The one or more remote computers 808 can include multiple computing devices, in various aspects. In aspects where the network 806 is distributed in configuration, the one or more remote computers 808 can be located at one or more different geographic locations. In an aspect where the one or more remote computers 808 are a plurality of computing devices, each of the plurality of computing devices can be located across various locations, such as buildings in a campus, medical and research facilities at a medical complex, offices or “branches” of a banking/credit entity, or can be mobile devices that are wearable or carried by personnel, or attached to vehicles or trackable items in a warehouse, for example. In some aspects, the remote computers 808 are physically located in a medical setting, such as, for example, a laboratory, inpatient room, an outpatient room, a hospital, a medical vehicle, a veterinary environment, an ambulatory setting, a medical billing office, a financial or administrative office, hospital administration setting, an in-home medical care environment, and/or medical professionals' offices. The remote computers 808 might also be physically located in nontraditional healthcare environments so that the entire healthcare community might be capable of integration on the network 806. In other aspects, the remote computers 808 can be physically located in a non-medical setting, such as a packing and shipping facility or deployed within a fleet of delivery or courier vehicles.

Continuing, the computing environment 800 includes a database 804. Although shown as a single component, the database 804 can be implemented using multiple databases that are communicatively coupled to one another, independent of the geographic or physical location of a memory device. The database 804 can, for example, store data in the form of artifacts, server lists, properties associated with servers, environments, properties associated with environments, computer instructions encoded in multiple different computer programming languages, deployment scripts, applications, properties associated with applications, release packages, version information for release packages, build levels associated with applications, identifiers for applications, identifiers for release packages, users, roles associated with users, permissions associated with roles, workflows and steps in the workflows, clients, servers associated with clients, attributes associated with properties, audit information, and/or audit trails for workflows. The database 804 can, for example, also store data in the form of electronic records, such as electronic medical records of patients, patient-specific documents and historical records, transaction records, billing records, task and workflow records, chronological event records, and the like. Generally, the database 804 includes physical memory that is configured to store information encoded in data. For example, the database 804 can provide storage for computer-readable instructions, computer-executable instructions, data structures, data arrays, computer programs, applications, and other data that supports the functions and actions to be undertaken using the computing environment 800 and components shown in the example of FIG. 8.

As shown in the example of FIG. 8, when the computing environment 800 operates with distributed components that are communicatively coupled via the network 806, computer instructions, applications, extensions, and/or program modules can be located in local and/or remote computer storage media (e.g., memory storage devices). Aspects of the present invention can be described in the context of computer-executable instructions, such as program modules, being executed by a computing device. Program modules can include, but are not limited to, routines, programs, objects, components, and data structures that perform particular tasks or implement particular abstract data types. In aspects, the computing device 802 can access, retrieve, communicate, receive, and update information stored in the database 804, including program modules. Accordingly, the computing device 802 can execute, using a processor, computer instructions stored in the database 804 in order to perform aspects described herein.

Although internal components of the devices in FIG. 8, such as the computing device 802, are not illustrated, those of ordinary skill in the art will appreciate that internal components and their interconnection are present in the devices of FIG. 8. Accordingly, additional details concerning the internal construction device are not further disclosed herein. Although many other internal components of the computing device 802 and the remote computers 808 are not shown, such components and their interconnection are known. Accordingly, additional details concerning the internal construction of the computing device 802 and the remote computers 808 are not further disclosed herein.

Additionally, it will be understood by those of ordinary skill in the art that the computing environment 800 is just one example of a suitable computing environment and is not intended to limit the scope of use or functionality of the present invention. Similarly, the computing environment 800 should not be interpreted as imputing any dependency and/or any requirements with regard to each component and combination(s) of components illustrated in FIG. 8. It will be appreciated by those having ordinary skill in the art that the connections illustrated in FIG. 8 are just examples as other methods, hardware, software, and devices for establishing a communications link between the components, devices, systems, and entities can be utilized in implementation of the present invention. Although the connections are depicted using one or more solid lines, it will be understood by those having ordinary skill in the art that the example connections of FIG. 8 can be hardwired or wireless, and can use intermediary components that have been omitted or not included in FIG. 8 for simplicity's sake. As such, the absence of components from FIG. 8 should not be interpreted as limiting the present invention to exclude additional components and combination(s) of components. Moreover, though devices and components are represented in FIG. 8 as singular devices and components, it will be appreciated that some aspects can include a plurality of the devices and components such that FIG. 8 should not be considered as limiting the number of a device or component.

The present invention has now been described in relation to particular aspects, which are intended in all respects to be illustrative rather than restrictive. Thus, the present invention is not limited to these aspects, but variations and modifications can be made without departing from the scope of the present invention.

Although the present technology has been described in detail for the purpose of illustration based on what is currently considered to be the most practical and preferred implementations, it is to be understood such detail is solely for that purpose and the technology is not limited to the disclosed implementations, but, on the contrary, is intended to cover modifications and equivalent arrangements that are within the spirit and scope of the appended claims. For example, it is to be understood the present technology contemplates that, to the extent possible, one or more features of any implementation can be combined with one or more features of any other implementation.

1. One or more non-transitory computer-readable media having computer-executable instructions embodied thereon that, when executed, perform a method for a sender service, the media comprising:
receiving a request to transmit an outgoing electronic communication and a designated clinical encounter;
automatically generating the outgoing electronic communication in response to the request, the outgoing electronic communication including the designated clinical encounter for review by a user; and
automatically transmitting the outgoing electronic communication including the designated clinical encounter, the outgoing electronic communication having an identifier that is associated with the designated clinical encounter.

2. The media of claim 1, wherein automatically generating the outgoing electronic communication comprises:
retrieving an electronic record of the designated clinical encounter, the electronic record of the designated clinical encounter including one or more digitized images; and
integrating the one or more digitized images with the outgoing electronic communication, wherein the one or more digitized images are integrated by attachment to the outgoing electronic communication or by insertion into a message body of the outgoing electronic communication.

3. The media of claim 2, wherein the one or more digitized images include one or more of:
a radiology image;
a nuclear radiology image;
a mammogram;
a Computerized Axial Tomography (CAT) image;
a Positron Emission Tomography (PET) image;
a Magnetic Resonance Image (MRI);
an x-ray image;
an ultrasound image;
a photograph;
a video;
a fluoroscopy image; or
a pathology specimen image.

4. The media of claim 1, wherein automatically generating the outgoing electronic communication comprises:
identifying the user who is associated with the designated clinical encounter, the user being a clinician; and
identifying an electronic address that is assigned to the user identified as being associated with the designated clinical encounter.

5. The media of claim 1, wherein automatically generating the outgoing electronic communication comprises:
identifying the user who is associated with a patient corresponding to the designated clinical encounter, the user being a clinician who is identified from an order requesting the designated clinical encounter of the patient and clinical findings from the user based on the designated clinical encounter; and
identifying an electronic address that is assigned to the user identified as being associated with the patient corresponding to the designated clinical encounter.

6. The media of claim 1, wherein automatically generating the outgoing electronic communication comprises:
generating a selectable link that, when selected, automatically directs the user to a secure web-based high-resolution image viewer for entry of credentials to login to the secure web-based high-resolution image viewer, wherein the secure web-based high-resolution image viewer enables display, subsequent to successful verification of the credentials entered, of one or more digitized images that correspond to the designated clinical encounter; and
integrating the selectable link with the outgoing electronic communication, wherein the selectable link is integrated by attachment to the outgoing electronic communication or by insertion into a message body of the outgoing electronic communication.

7. The media of claim 1, further comprising:
prior to transmitting the outgoing electronic communication, automatically scrubbing Patient Identifying Information (PII) from the designated clinical encounter.

8. The media of claim 7, further comprising,
integrating a unique identifier in the outgoing electronic communication, wherein the unique identifier specifically corresponds to the designated clinical encounter, and wherein the unique identifier does not include PII.

9. The media of claim 1, wherein the request includes a selection of one clinical encounter, and wherein the one clinical encounter becomes the designated clinical encounter based on the selection.

10. One or more non-transitory computer-readable media having computer-executable instructions embodied thereon that, when executed, perform a method for a receiver service, the media comprising:
for each one of a plurality of designated clinical encounters:
receiving a first indication of a request to transmit an outgoing electronic communication and a designated clinical encounter, the designated clinical encounter including one or more digitized images captured for a clinical encounter of a patient;
in response to receiving the first indication, generating a unique identifier for the designated clinical encounter, the unique identifier being free of Patient Identifying Information (PII);
assigning the unique identifier to the designated clinical encounter;
storing an association of the designated clinical encounter and the unique identifier assigned to the designated clinical encounter in memory;
receiving a second indication of the outgoing electronic communication and the designated clinical encounter being transmitted;
in response to the second indication, monitoring for an incoming electronic communication that includes the unique identifier assigned to the designated clinical encounter;
based on the monitoring, receiving the incoming electronic communication from a first clinical user that includes the unique identifier assigned to the designated clinical encounter in the memory, the incoming electronic communication including one or more clinical findings from the first clinical user for the designated clinical encounter;
extracting the unique identifier from the incoming electronic communication;
mapping the unique identifier to the designated clinical encounter based on the association stored in the memory in order to identify an electronic record of the designated clinical encounter corresponding to the patient; and
automatically modifying the electronic record of the designated clinical encounter to include the one or more clinical findings from the first clinical user for the designated clinical encounter corresponding to the patient from the incoming electronic communication.

11. The media of claim 10, further comprising:
subsequent to modifying the electronic record of the designated clinical encounter to include the one or more clinical findings, deleting the association of the designated clinical encounter and the unique identifier from the memory.

12. The media of claim 10, further comprising:
identifying a second clinical user who is associated with the designated clinical encounter corresponding to the patient and an electronic address of the second clinical user;
generating a notification to be transmitted to the electronic address of the second clinical user, the notification including the one or more clinical findings from the first clinical user for the designated clinical encounter corresponding to the patient; and
transmitting the notification to the second clinical user.

13. The media of claim 12, wherein the second clinical user has placed a medical order for the clinical encounter of that patient, and wherein the first clinical user is another clinician from whom interpretation of the clinical encounter is being requested from the second clinical user.

14. The media of claim 10, further comprising, based on the monitoring:
receiving a plurality of incoming electronic communications;
scanning each of the plurality of incoming electronic communications for the unique identifier; and
recognizing one of the plurality of incoming electronic communications is the incoming electronic communication that includes the unique identifier assigned to the designated clinical encounter in the memory.

15. The media of claim 10, wherein mapping the unique identifier to the designated clinical encounter based on the association stored in the memory comprises:
searching the memory for the unique identifier;
identifying that the unique identifier is assigned to the designated clinical encounter in the memory; and
locating the electronic record that corresponds to the designated clinical encounter.

16. The media of claim 10, wherein a plurality of unique identifiers are generated and assigned to the plurality of designated clinical encounters, and are stored in association with the plurality of designated clinical encounters in the memory.

17. The media of claim 16, wherein the association of the plurality of unique identifiers with the plurality of designated clinical encounters are individually deleted from the memory when the plurality of unique identifiers are individually mapped as a plurality of incoming electronic communications are individually received.

18. The media of claim 10, wherein the incoming electronic communication is an externally-generated email.

19. The media of claim 10, wherein the incoming electronic communication is received from a secure web-based high-resolution image viewer.

20. A system comprising:
a database that stores a mapping of a plurality of unique identifiers to a plurality of designated clinical encounters;
a computing device coupled to the database, the computing device comprising a processor, a memory, a first service, and a second service, wherein the processor executes computer-executable instructions in the memory that operate the first service and the second service;
wherein the first service:
receives a request to transmit an outgoing electronic communication and a designated clinical encounter, the designated clinical encounter including one or more digitized images captured for a clinical encounter of a patient;
automatically generates the outgoing electronic communication in response to the request, the outgoing electronic communication including the designated clinical encounter for review by a first clinical user; and
automatically transmits the outgoing electronic communication, including the designated clinical encounter to the first clinical user, the outgoing electronic communication having a unique identifier that is associated with the designated clinical encounter; and
wherein the second service:
receives an indication of the request to transmit the outgoing electronic communication and the designated clinical encounter;
in response to receiving the indication, generates the unique identifier for the designated clinical encounter, the unique identifier being free of Patient Identifying Information (PII);
assigns the unique identifier to the designated clinical encounter;
stores an association of the designated clinical encounter and the unique identifier assigned to the designated clinical encounter in the memory;
receives a second indication of the outgoing electronic communication and the designated clinical encounter being transmitted;
in response to the second indication, monitors for an incoming electronic communication that includes the unique identifier assigned to the designated clinical encounter;
based on the monitoring, receives the incoming electronic communication from the first clinical user that includes the unique identifier assigned to the designated clinical encounter in the memory, the incoming electronic communication including one or more clinical findings from the first clinical user for the designated clinical encounter;
extracts the unique identifier from the incoming electronic communication;
maps the unique identifier to the designated clinical encounter based on the association stored in the memory in order to identify an electronic record of the designated clinical encounter corresponding to the patient; and
modifies the electronic record of the designated clinical encounter to include the one or more clinical findings from the first clinical user for the designated clinical encounter corresponding to the patient from the incoming electronic communication.
